(19) United States
(12) Patent Application Publication
(10) Pub. No.: US 2023/0166122 A1
(43) Pub. Date: Jun. 1, 2023

(54) MULTI-FUNCTION THERAPEUTIC SYSTEM AND ASSOCIATED METHOD

(71) Applicant: Ammortal Inc., Baltimore, MD (US)

(72) Inventors: Brian Le GETTE, Voorhees, NJ (US); Steven YOUNG, Voorhees, NJ (US)

(21) Appl. No.: 17/843,814

(22) Filed: Jun. 17, 2022

Related U.S. Application Data
(60) Provisional application No. 63/285,057, filed on Dec. 1, 2021.

(51) Int. Cl. A61N 5/06; A61N 2/02

(52) U.S. Cl. CPC A61N 5/06; A61N 2/02; A61N 2005/0658

(57) ABSTRACT

A multi-function therapeutic system comprising a housing having a top portion having a first radiation therapy unit mounted therein for generating and applying light energy to a person, a bottom portion including a hydrogen therapy unit for supplying hydrogen to the person, and an intermediate bed portion disposed between the top portion and bottom portion. The intermediate bed portion includes an electromagnetic therapy unit for generating and applying electromagnetic energy to the person. The system can also include a vibration therapy unit associated with one or more of the bottom portion and the intermediate bed portion for generating and applying vibrational energy to the person, and a control unit for controlling the first radiation therapy unit, the hydrogen therapy unit, the electromagnetic therapy unit, and the sound therapy unit.

### RELATED APPLICATION

The present application claims priority to U.S. provisional patent application Ser. No. 63/285,057, filed on Dec. 1, 2021, and entitled Multi-Function Therapeutic Device, the contents of which are herein incorporated by reference.

### BACKGROUND OF THE INVENTION

The present invention is directed to therapeutic and other devices and associated systems, and more specifically is directed to therapeutic devices for applying various types of therapeutic techniques and energy to a person.

Today's world places numerous stresses on the human body, including toxins from our environment, unhealthy low nutrition foods, lack of physical movement, poor gut health, and psychological and emotional stress. These all combine to cause destructive damage to the body and its systems. These accumulated stresses disrupt a person's natural systems knocking them out of equilibrium, health and wellness. This can give rise to adverse bodily consequences, including the multiple forms of often debilitating chronic disease. The result is slower healing, accelerated aging, manifestation of illnesses, poor concentration and memory, fatigue, chronic anxiety, and poor sleep quality.

Traditional approaches to these problems involve pharmaceuticals, surgery, psycho-behavioral therapy and other allopathic methods. However, these methods often do not address the underlying cause but rather just treat the symptoms. Further, people may try other traditional remedies, one at a time, with limited if any success.

### SUMMARY OF THE INVENTION

The present invention is directed to a system and associated method for applying multiple different therapeutic techniques and enhancement technologies and protocols to a person or subject while lying or sitting in a housing shaped as a therapy bed, seat or lounger. The therapy bed is configured to be able to apply multiple different therapeutic techniques to the person in any selected sequence, at any selected intensity level, in any selected combination, and for any selected duration of time. The therapeutic techniques can be alternated or can be continuously applied to the subject during a selected therapeutic session. The therapy bed can include, for example, any selected combination of a hydrogen therapy unit for supplying molecular hydrogen to the subject, an electromagnetic unit for applying electromagnetic energy, a radiation unit for applying light energy, a vibration therapy unit for applying vibrational energy, and an acoustic therapy unit for applying audio instruction to the subject (e.g., meditation exercises) and to supply music. The acoustic therapy unit and the vibration therapy unit can share selected hardware components or can be completely separate units.

The various different therapeutic techniques applied by the different therapy units serve to provide healing energy to the subject and enable the delivery of therapeutic guidance, psychoacoustic therapy and/or music therapy, such as guided meditation, guided breathwork and binaural beats. The many different techniques, combination of techniques, or other combination protocols to be applied by the therapy bed can be preprogrammed, can be part of multi-session protocols, or can be selected in real time by the subject, their therapist, their clinician, or other person either prior to or during a therapeutic session. The therapeutic session can last for any selected period of time deemed sufficient for the subject to be exposed to the therapeutic effects of the applied therapeutic techniques.

The present invention is directed to a multi-function therapeutic system comprising a housing having a top portion having a first radiation therapy unit mounted therein for generating and applying light energy to a person, a bottom portion including a hydrogen therapy unit for supplying hydrogen to the person, and an intermediate bed portion disposed between the top portion and bottom portion. The intermediate bed portion includes an electromagnetic therapy unit for generating and applying electromagnetic energy to the person. The system can also include a vibration therapy unit associated with one or more of the bottom portion and the intermediate bed portion for generating and applying vibrational energy to the person, and a control unit for controlling the first radiation therapy unit, the hydrogen therapy unit, the electromagnetic therapy unit, and the sound therapy unit. Further, the bottom portion, the bed portion and/or the top portion can include an acoustic therapy unit for generating an audio output. The bottom portion can also include a second radiation therapy unit for generating and applying light energy to the person.

The vibration therapy unit can include a first vibration generating sub-system coupled to the bottom portion for generating a first vibrational energy and a second vibrational subsystem coupled to the intermediate bed portion for generating a second vibrational energy. The first vibration generating sub-system includes a bass element, such as a sub-woofer, and the second vibration generating sub-system includes one or more transducers. The vibration therapy unit can optionally be configured to operate as an acoustic therapy unit to generate an audio output. Further, the first and/or second radiation therapy units can generate light energy in a blue wavelength range, a red wavelength range, or an infrared wavelength range. Regarding the infrared wavelength range, the first and second radiation therapy units can generate light energy in one or more of a near-infrared wavelength range, a mid-infrared wavelength range, and a far-infrared wavelength range.

Further, the electromagnetic therapy unit can generate the electromagnetic energy in a continuous or a non-continuous manner. The electromagnetic therapy unit can include one or more pulsed electromagnetic field (PEMF) coils for generating the electromagnetic energy. According to one embodiment, the controller can control movement of the top portion relative to the bottom portion between an open position and a closed position. Also, the top portion can be coupled to the bottom portion via a support stanchion, where the support stanchion includes an outer support element and an inner support element disposed within the outer support element. The inner support element is configured for vertical movement within the outer support element.

According to another embodiment, the present invention us directed to a multi-function therapeutic system comprising a housing having a top portion having a first radiation therapy unit mounted therein for generating and applying radiation energy to a person, a bottom portion, a bed portion supported by the bottom portion, and a control unit. The bottom portion can include a hydrogen therapy unit for supplying hydrogen to the person, and a second radiation therapy unit for generating and applying radiation energy to the person, and the control unit can be configured to control the first radiation therapy unit, the second radiation therapy unit, and the hydrogen therapy unit.

The bed portion can include an electromagnetic therapy unit for generating and applying electromagnetic energy to the person. The bottom portion can further include a vibration therapy unit for generating and applying vibrational energy to the person.

According to still other embodiments, the present invention is directed to a method for applying one or more therapeutic techniques to a person. The method comprises providing a housing having a bottom portion, a top portion, and a bed portion disposed between the bottom portion and the top portion, providing a first radiation therapy unit mounted in the top portion for generating and applying light energy to a person, providing in the bottom portion a hydrogen therapy unit for supplying hydrogen to the person, and a vibration therapy unit for generating and applying vibrational energy to the person, providing in the bed portion an electromagnetic therapy unit for generating and applying electromagnetic energy to the person, and controlling the first radiation therapy unit, the hydrogen therapy unit, the electromagnetic therapy unit, and the sound therapy unit. The bottom portion can include a second radiation therapy unit for generating and applying light energy to the person.

The method can also include generating with one or more of the first and second radiation therapy units light energy in one or more of a near-infrared wavelength range, a mid-infrared wavelength range, or a far-infrared wavelength range, and controlling movement of the top portion relative to the bottom portion between an open position and a closed position. Further, the bottom portion and/or the top portion can include at least a portion of an acoustic therapy unit for generating an audio output.

The present invention can also be directed to a multi-function therapeutic system comprising a housing having a top portion having a first radiation therapy unit mounted therein for generating and applying light energy to a person; a bottom portion including a second radiation therapy unit mounted therein for generating and applying light energy to the person where the bottom portion supports a transparent bed portion having an electromagnetic therapy unit associated therewith for generating and applying electromagnetic energy to the person via one or more coils; and a control unit for controlling the first radiation therapy unit, the second radiation therapy unit, and the electromagnetic therapy unit. The bed portion is spatially separated from the base portion to form an intermediate bed portion disposed between the base portion and the top portion.

The housing can be configured to further include one or more of a hydrogen therapy unit for supplying hydrogen to the person, and a vibration therapy unit for generating and applying vibrational energy to the person. The housing can also include an acoustic therapy unit for generating an audio output. The first and second radiation therapy units can generate light energy in one or more of a near-infrared wavelength range, a mid-infrared wavelength range, and a far-infrared wavelength range. The electromagnetic therapy unit generates the electromagnetic energy in a continuous or a non-continuous manner and can include one or more pulsed electromagnetic field (PEMF) coils for generating the electromagnetic energy. Further, the controller controls movement of the top portion relative to the bottom portion and the bed portion between an open position and a closed position.

The present invention can be further directed to a multi-function therapeutic system comprising a housing having an acoustic therapy unit for providing an audio output, a vibration therapy unit for generating and applying vibrational energy to a person, and at least two or more of: a first radiation therapy unit for generating and applying light energy to the person, a second radiation therapy unit for generating and applying light energy to the person, an electromagnetic therapy unit for generating and applying electromagnetic energy to the person, and a hydrogen therapy unit configured to supply hydrogen to the person. The audio output of the acoustic therapy unit comprises one or more of operating instructions for the system, providing mediation instructions for the person, providing breathing exercise instructions, and providing micro-movement therapy instructions.

According to still other embodiments, the present invention is directed to a multi-function therapeutic system comprising a housing having a transparent bed portion forming a support surface for a person where the bed portion has a main body having one or more coils coupled thereto for generating and applying electromagnetic energy to the person. The coils can include one or more wires having an insulating layer formed over at least a portion of the one or more wires. The system can also include a base portion having one or more support elements for supporting the transparent bed portion and having a first radiation therapy unit for generating and applying light energy to the person. The light energy passes through the transparent bed portion to the person.

The housing can further include a top portion that is movable relative to one or more of the bed portion and the base portion. The top portion includes a second radiation therapy unit for generating and applying light energy to the person. The first radiation therapy unit generates light energy in one or more of a near-infrared wavelength range, a mid-infrared wavelength range, and a far-infrared wavelength range, and the coils can include one or more pulsed electromagnetic field (PEMF) coils for generating the electromagnetic energy. The main body of the transparent bed portion can include a first bed portion, a second bed portion and a third bed portion, where the second bed portion is disposed between and is angled relative to the first and third bed portions forming multiple different planes.

The present invention can be further directed to a method of forming a transparent bed portion of a multi-function therapeutic system by embedding or coupling at least one electromagnetic field generation coils relative to a support surface of the transparent bed, forming at least one groove in the support surface that are sized and configured for seating one or more wires of the one or more electromagnetic field generation coils, inserting the wire in the groove to form a friction fit, applying a selected hardening material to the groove to secure the wire in the groove, heating one or more portions of the support surface to a selected temperature such that the heated portion is pliable, and bending the one or more heated portions of the support surface to form one or more angled sections. The system can include one or more transducers that are coupled to the support surface to generate vibrational energy, and/or one or more lights coupled to the support surface. A protective or insulative sheathing can be provided on the wire prior to insertion into the groove.

### BRIEF DESCRIPTION OF THE DRAWINGS

These and other features and advantages of the present invention will be more fully understood by reference to the following detailed description in conjunction with the attached drawings in which like reference numerals refer to like elements throughout the different views. The drawings illustrate principals of the invention and, although not to scale, show relative dimensions.

FIG. 1 is a schematic block diagram illustration of the various therapy applying units forming the multi-function therapeutic system of the present invention.

FIG. 2 is a schematic block diagram showing the multi-function therapeutic system configured as a therapy bed according to the teachings of the present invention.

FIGS. 3 and 4 are perspective views of one embodiment of the therapy bed according to the teachings of the present invention.

FIG. 5 is a partial cross-sectional view of the base portion of the therapy bed according to the teachings of the present invention.

FIG. 6A is a perspective view of the intermediate bed portion of the therapy bed according to the teachings of the present invention.

FIG. 6B is a partial cross-sectional view of the bed portion of the therapy bed showing the wire grooves formed therein according to the teachings of the present invention.

FIG. 7 is a front view of the therapy bed showing the top portion positioned in the open position according to the teachings of the present invention.

FIG. 8 is a front view of the therapy bed showing the top portion positioned in the closed position according to the teachings of the present invention.

### DETAILED DESCRIPTION

The present invention is directed to a multi-function therapeutic system and associated method for combining, in a synergistic manner, multiple different therapeutic (e.g., energetic, enhancing and restorative) technologies to beneficially repair, restore, and regenerate important systems and portions of the human body.

As used herein, the term “therapeutic unit” or “therapy unit” or “therapeutic techniques” is intended to include any device, system or subsystem that is capable of providing or applying any combination of a fluid (e.g., breathable gas), radiation energy, electromagnetic energy, audio information, music, audio guided therapy, acoustic energy, vibrational energy, hot or cold temperature, negative ions, air pressure, or other type of energy, to a subject, and which has a beneficial effect on the subject or person. The fluid can include molecular hydrogen or any other suitable gas. The radiation energy can include light energy in any selected wavelength range, including visible light and non-visible light.

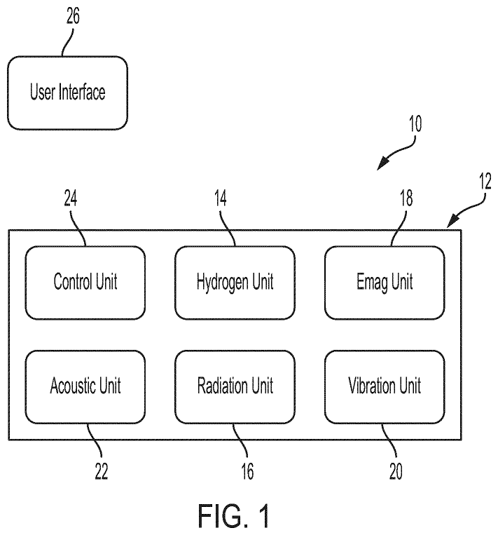

As shown in FIG. 1, the present invention is directed to a multi-function therapeutic system 10 that includes a housing 12 that includes a plurality of different healing and restorative therapy units, such as for example a hydrogen therapy unit 14 for applying or supplying hydrogen to a subject. According to one practice of the present invention, the illustrated hydrogen therapy unit 14 can supply hydrogen, such as molecular hydrogen, to the subject to help the cells of the subject's body and to minimize deterioration thereof. In another practice, the hydrogen therapy unit 14 can supply a gas composed of two parts hydrogen and one part oxygen. Hydrogen is a powerful antioxidant that helps ameliorate or reduce the negative and harmful inflammatory effects on the body. Hydrogen can help reduce inflammation at the cellular level, help protect organs from damage, maximize the metabolism of the body, help restore brain function, and help reduce obesity and blood sugar dysfunction. The benefits of the hydrogen therapy unit 14 can be further enhanced when used in connection with other types of therapies, such as those applied by the therapy units described herein.

The illustrated multi-function therapeutic system 10 can also employ a radiation therapy unit 16 for generating and controlling a subsystem that includes one or more radiation panels for applying radiation or light energy to the subject to promote a beneficial effect. The radiation therapy unit 16 can generate light energy in one or more wavelength ranges. For example, and according to one embodiment, the radiation therapy unit 16 can generate radiation, including light energy, in many different wavelengths, including in one or more of the blue (e.g., between about 380 nm and about 500 nm), red (e.g., between about 600 nm and about 700 nm) and infrared (e.g., between about 700 nm and 10,000 nm) wavelength ranges. The infrared light energy can include light energy in one or more of the near-infrared (NIR or IR-A) (e.g., between about 700 nm and about 1,400 nm) wavelength range, the mid-infrared (MIR or IR-B) (e.g., between about 1,400 nm and about 3,000 nm) wavelength range, and the far-infrared (FIR or IR-C) (e.g., between about 3,000 nm and 10,000 nm) wavelength range. Specifically, the radiation therapy unit 18 can include a series of radiation emitters, such as for example light emitting diodes (LEDs), that can be arranged in any selected pattern or sequence. The emitters preferably emit radiation or light energy in the near infrared, mid infrared, or far infrared wavelength ranges, or any combination thereof, and can emit radiation at a single frequency, multiple different frequencies or the frequency can be adjustable. The light energy can help the cells of the subject's body heal by stimulating energy production in the cells. The light energy can also stimulate the healing of the skin to reduce wrinkles, balance the fat to muscle ration in the body, stimulate the production of sex hormones, and optimize or enhance sleep quality. The light energy benefits can be further enhanced when used in connection with one or more of the other types of therapy units.

The multi-function therapeutic system 10 can also include an electromagnetic therapy unit 18 for selectively generating and applying electromagnetic energy to the subject in order to promote a beneficial effect. The electromagnetic therapy unit 18 can generate and apply the electromagnetic energy to the subject in a continuous or non-continuous manner, and at a single frequency or at multiple different frequencies. According to one embodiment, the electromagnetic therapy unit 18 can employ one or more coils, such as for example pulsed electromagnetic field (PEMF) coils, for applying electromagnetic energy to the subject, at one or more frequencies simultaneously or sequentially. The electromagnetic energy translates at a cellular level, in which it enhances the body's overall functioning. The process of receiving electromagnetic energy involves sending electromagnetic energy waves towards specific areas of the body or generally to the entire body. The electromagnetic waves pass completely through the body and increase the spin rate of the electrons in the cells. As a result, wellness levels are improved and the body functions more efficiently. Further, electromagnetic energy serves to enhance the cells ability to absorb energy from other energy sources. The electromagnetic energy can also help enhance bone recovery, decrease anxiety and depression, regenerate nerves, and regrow cartilage in the body. The electromagnetic energy can be intermittent (e.g., pulsed) or can be continuously applied to the person for a selected period of time.

Still further, the multi-function therapeutic system 10 can employ a vibration unit 20 for generating and applying various forms of sound vibrational energy, including sound vibrational energy well below the hearing range of the subject to the subject to promote a beneficial effect. The vibration therapy unit 20 can include one or bass modules (e.g., subwoofers) and one or more speakers or transducers for generating the vibrational energy. The vibration therapy unit 20 can employ multiple channels to enable the vibrational energy to be directed to one or more specific areas of the subject's body directly or through any intervening surface. The vibrational energy can help the subject's body heal. Specifically, the vibration therapy unit 20 can produce or generate sound waves to cause or create vibrational energy in a surface and hence the subject. The vibrational energy can serve to help alter moods, heal bones, activate genes for healing, and calm the nervous system of the subject. The vibrational energy can also serve to directly alter the psychological state of the subject and directly improve the overall wellbeing thereof.

The illustrated multi-function therapeutic system 10 can also include additional therapy units, such as an acoustic therapy unit 22, for generating an audio output for psycho-behavioral therapy, music, voice, and voice guidance and voice therapy. The acoustic therapy unit 22 can be a unit separate from the vibration therapy unit 20, can share one or more selected components (e.g., sub-woofer, speakers, transducers, power source, and the like) with the vibration therapy unit 20, or can be formed as a single unit that can be controlled to perform different functions, such as by providing vibroacoustic energy or psychoacoustic audio output. For ease of illustration, the vibration therapy unit 20 and the acoustic therapy unit are shown as separate units, although they can share common components. The audio output can include selected types of music, voice or sound to promote a beneficial effect. The acoustic therapy unit 22 can include one or more speakers and transducers for generating the audio output. The acoustic unit 22 can have multiple channels to enable the sound energy to create three-dimensional sound quality and/or direct the sounds impact to the subject's ears, to another area of the user's body, or another location. The acoustic unit can allow for multiple voices, musical compositions, and other audio signals to be blended from one or more audio tracks. The music output can help improve or affect behavior, motor function, social skills, emotions, coordination, self-expression and personal growth. Specifically, the music therapy can help reduce anxiety and physical effects of stress, improve healing, reduce depression and other symptoms in the elderly, and help reduce symptoms of selected psychological disorders. Further, the acoustic therapy unit 22 can provide an audio output to help guide the subject through one or more meditative sessions or exercises.

The multi-function therapeutic system 10 can also include a controller or control unit 24 for controlling the therapy units and any additional units in the system 10. The control unit 24 can be programmed to activate any of the selected therapy units, in any selected sequence, for any selected duration of time according to or based on the length of the therapy session. Specifically, the therapy units can be activated by the control unit 24 so as to apply selected therapy energy to the subject in any selected order or in any selected combination, for any selected length of time. The therapy units can be continuously or non-continuously applied to the subject. The control unit 24 can include any suitable types of hardware, such as a processor and memory, and any suitable software, for operating the therapy units of the system 10. The control unit 24 can be directly programmed or can receive downloaded programming via the Internet or other networked type systems, including operational system or unit programming, and various forms of content, including audio and vibrational content. The control unit 24 can connect directly or wirelessly to measurement equipment to test the system, to any therapy unit, to any system component for operating performance measurement, including functional performance, such as electrical performance and/or mechanical performance of the system, to one or more units or unit components using alarms or other alert triggers that can be sent wirelessly using a smartphone or other types of electronic devices. The control unit 24 can also employ simple and sophisticated data science calculations, including the use of machine learning and artificial intelligence techniques, to tune the system, including to an individual subject. In one embodiment, repeat use by a subject can customize the system to that specific subject and in another embodiment, real-time feedback can immediately customize the system to the subject.

The multi-function therapeutic system 10 can also include a user interface element 26 that can function as a display. The user interface element can generate one or more user interfaces for allowing the subject to interact with and control the operation of one or more portions of the multi-function therapeutic system 10 and can further provide an interface for allowing the user to select or program options for the system, such as additional functions of the therapy units. The user interface element can be coupled to the system, and hence to the therapy units, by any selected type of connection, such as by a wired or wireless connection, as is known in the art. More specifically, the user interface element can form part of the therapy bed or can be remote therefrom. In one embodiment, the user interface element 26 can include a touchscreen that can be fixed to or mechanically removable from the system, can be a wall mounted display, can employ any other types of holder elements, or can be configured as a handheld or other type of portable unit. The user interface element can be any selected types of display element, such as a touch screen display element. The control unit 24 and/or the interface element 26 can be used to create subject personalization including real-time system customization based on subject biofeedback and other subject measurement tools. The biofeedback or additional types of subject performance and system measurement instruments, such as devices that measure heart rate variability, bioluminescence, galvanic skin response, breath rate, heart rate and other subject performance measures, can be in communication with or integrated with the system, or can be optionally attached to the system, or can be separate therefrom. The user interface element 26 can also be employed to communicate with and control other location elements that are separate and distinct from the multi-function therapeutic system of the present invention. For example, the user interface element can be used to control shades or blinds in a room as well as control one or more light sources to adjust the light levels. The user interface element can also be used to control one or more aromatherapy units, one or more fans, one or more air cleaners, one or more ozone producing units, one or more negative ion producing units, audio equipment, white noise equipment, and any other electronically controllable system. The user interface element or a separate device can also be used to receive visual, sensory or auditory input so as to control, program or otherwise customize the system or any individual therapy unit, such as by using voice analysis to program the radiation therapy unit, so as to pulse and apply near infrared light to the subject.

The multi-function therapeutic system 10 of the present invention is a combination of one or more of the foregoing energetic technologies, and specifically applies a combination of multiple healing and/or longevity based technologies in a single system or device to enable the use, either simultaneously or in any combination or adaptive sequence, of the technologies.

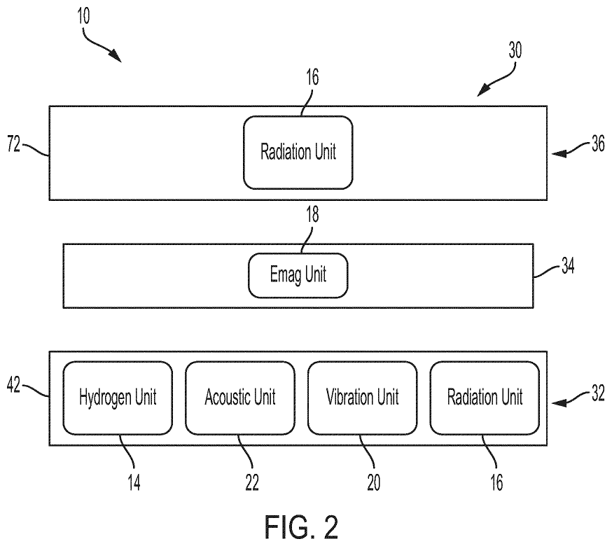

The multi-function therapeutic system 10 of the present invention can be in the shape or form of a therapy bed having multiple discrete components. The therapy bed can be configured as a flat or angled bed, a seat or chair, or as a lounger. For example, as shown schematically in FIG. 2, the therapy bed can have a general housing 30 that has multiple distinct components. The housing components can include a base portion 32 for housing one or more therapy units, a top portion 36 that can move relative to the base portion 32 and is configured to house one or more therapy units, and an optional bed portion 34 that is disposed between the base portion 32 and the top portion 36 or can form part of the base portion 32. That is, the bed portion 34 can be supported by the base portion 32 via suitable support structure, and hence can be deemed to form part of the base portion 32. Alternatively, the bed portion 34 can be disposed between the base portion 32 and the top portion 36 to form an intermediate bed portion. The bed portion 34 can include, if desired, one or more optional therapy units. The base portion 32, the top portion 36, and the bed portion 34 can be mechanically and operatively coupled together. The top portion, bed portion and/or base portion can move relative to each other in many different ways, including for example by pivoting or sliding movement and/or by any other types of movement that separate the top portion from the base portion and/or bed portion.

According to one embodiment, the base portion 32 can include a housing 42 that can mount or house one or more therapy units and support, if desired, the bed portion 34. Specifically, the housing 42 can mount any selected combination of the therapy units. According to one embodiment, the housing 42 can mount the hydrogen therapy unit 14, which can include a water gauge, water fill pipe, and hydrogen gas port, for supplying hydrogen to the subject when using the therapy bed. The base portion can also optionally include any combination of the acoustic therapy unit 22, the vibration therapy unit 20, and the radiation therapy unit 16. The top portion 36 of the therapy bed can optionally include a radiation unit 16 or components thereof and optionally one or more components, such as speakers, of the acoustic therapy unit 22. As such, and as configured, either or both of the base portion 32 and the top portion 36 can include the radiation unit 16. In one embodiment, the optional intermediate bed portion 34 can include one or more component parts of or all of the electromagnetic unit 18, and can include one or more component parts of the vibration therapy unit 20 and the acoustic unit 22.

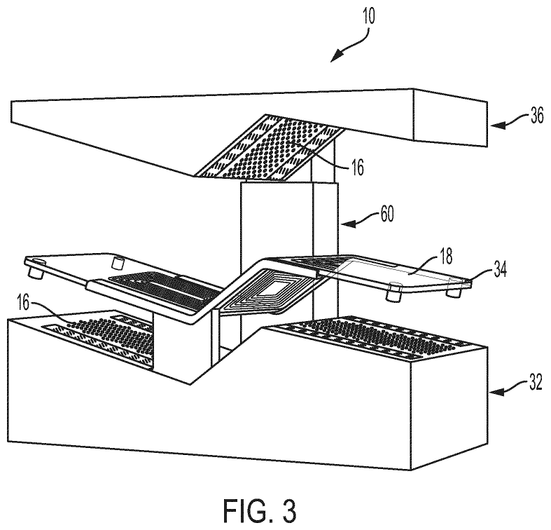

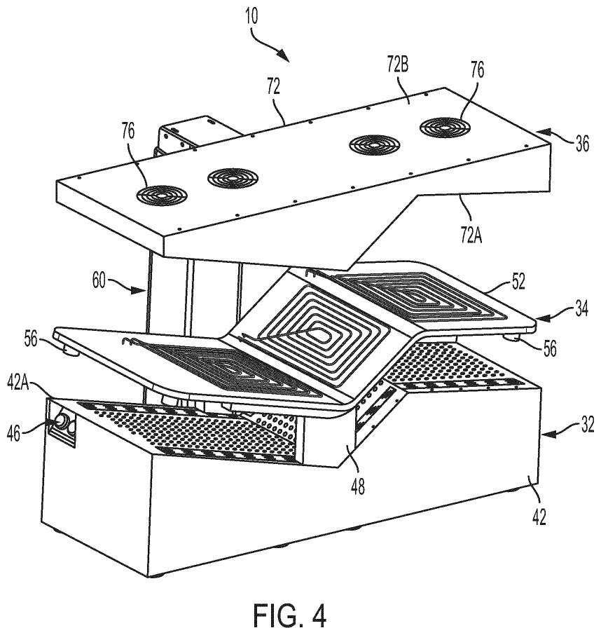

FIGS. 3-8 illustrate one embodiment of the multi-function therapeutic system 10 according to the teachings of the present invention. The multi-function therapeutic system 10, as noted, can be configured as a therapy bed. As shown in FIGS. 3 and 4, the top portion can include a housing 72 for mounting one or more radiation therapy units 16 for applying radiation to the subject when lying or sitting in the therapy bed. Specifically, the housing 72 can have multiple sides and a top and bottom surface forming an interior chamber. The bottom surface 72A facing the bed portion 34 can have mounted therein the radiation therapy unit 16. The radiation unit 16 can include a series of LEDs for generating radiation energy in specific wavelengths and wavelength bands or ranges. According to one embodiment, the radiation therapy unit in the top portion 36 can include selected LEDs for generating radiation or light energy in the blue, red, and infrared radiation ranges. The radiation therapy unit 16 can hence include LEDs configured for emitting energy in one of these ranges or in any combination of these ranges. For example, in the current embodiment, the LEDs can be configured to emit radiation or light energy in one or more of the red or infrared ranges, including the near infrared, mid infrared, far infrared wavelength ranges. The light energy can help the cells of the subject's body heal by stimulating energy production in the cells. The light energy can also stimulate the healing of the skin to reduce wrinkles, balance the fat to muscle ration in the body, stimulate the production of sex hormones, and optimize or enhance sleep quality. The LEDs of the radiation therapy unit 16 can be arranged in any selected pattern, and in any selected concentration, and can be continuous or pulsed or a combination of the two. For example, the LEDs emitting NIR light can be pulsed rather than powered continuously so as to aid in the recovery of the subject. When the radiation therapy unit 16 employs different types of LEDs for generating light energy in different wavelengths, the LEDs can also be arranged in any selected pattern. For example, the different LEDs can be interspersed in whole or in part with each other in a grid arrangement. Alternatively, the different LEDs in the grid arrangement can be concentrated, wholly or partially, in different sections. The LEDs can also be individually adjusted to emit light at different frequencies. The top surface 72B of the housing 72 can also include one or more cooling units 76, such as fans, for providing cooling and ventilation to the interior chamber and for cooling the radiation therapy unit 16.

The bottom portion 32 of the therapy bed can also include one or more types of therapy units. For example, in the current embodiment and as shown in FIGS. 3-5, the housing 42 of the bottom portion 32 can include a number of sides and has a top surface 42A and an opposed bottom surface, which in combination define an interior chamber 44. The chamber 44 can mount different types of therapy units, and the top surface 42A can also include one or more therapy units. Alternatively, the top surface can form or define a bed portion for supporting the subject when using the system. As illustrated, the chamber 44 of the bottom portion can include the hydrogen therapy unit 14. The hydrogen therapy unit 14 can generate hydrogen, such as molecular hydrogen (H₂), from a source fluid, such as water. The water can be converted into molecular hydrogen by one or more known processes or techniques, such as by PEM type fuel cells, ionization techniques, and the like. The molecular hydrogen is an agent with potential applications in oxidative stress-related and/or inflammatory disorders. The hydrogen can be administered or supplied to the subject by inhaling hydrogen-containing air (HCA) or by the oral intake of hydrogen-rich water (HRW). Alternatively, the base portion can house a hydrogen tank for supplying the molecular hydrogen without the need to generate the hydrogen in real time. The hydrogen therapy unit 14 can have a port interface 46 formed in one side of the housing 42 that allows the source fluid (e.g., water) to be added to the hydrogen therapy unit 14 and for the hydrogen to be extracted from the unit. Alternatively, the port interface 46 can include just a supply port if a hydrogen tank is provided. According to one embodiment, a cannula (not shown) can be coupled to the supply portion of the port interface 46 at one end and to the subject (or near the subject) at the other end for supplying the hydrogen supplied by the hydrogen therapy unit 14. A gauge for measuring water or gas levels can also be included, with or without a refill alarm system, and a water fill port can be co-located with the supply port or located anywhere else on the base portion. The molecular hydrogen (H₂) gas is a neutrally charged molecule that has the ability to cross cell membranes and the blood-brain barrier. This gives the molecular hydrogen the ability to access DNA and mitochondria within the cell where it initiates a protective cellular action.

The chamber 44 of the bottom portion 32 can also optionally mount a portion of the electromagnetic therapy unit 18, such as the power source and the like, for providing power to the electromagnetic coils. The coils or any other suitable metal structure associated with the electromagnetic therapy unit 18 can be mounted in the top surface 42A of the bottom portion 32 or can be provided in the bed portion 34. The coils can serve as a part of an electromagnetic circuit or as one or more antennas to generate one or more electromagnetic fields. Further, the chamber 44 can also optionally mount part or all of the vibrational therapy unit 20 for generating vibrational energy. Further, the chamber 44 can also optionally mount all or a portion of the vibration therapy unit 20 for generating vibrational energy. The vibration therapy unit 20 can produce or generate sound waves to cause or create vibrational energy in a surface of the therapy bed. The vibrational energy is then transferred through the associated mechanical components to the subject when lying in the therapy bed. The vibrational energy can serve to help alter moods, heal bones, activate genes for healing, and calm the nervous system of the subject. The vibrational energy can also serve to directly alter the psychological state of the subject and directly improve the overall wellbeing thereof. In one embodiment, the base portion 32 can mount the subwoofer for generating vibrations in the housing 42. The vibrations in the base portion 32 can be transmitted to the bed portion 34 by the support structure that couples the bed portion 34 to the base portion 32. Additionally, the bed portion 34 can also include one or more transducers 56 that form part of the vibration therapy unit 20 for generating and transmitting vibrational energy directly or indirectly to the bed portion and hence to the person. As such, the vibrational energy can be generated by the subwoofer in the base portion 32 and/or the transducers of the bed portion 34. According to an alternate embodiment, the subwoofer can be employed alone to generate the vibrational energy or the transducers 56 can be employed in addition to the subwoofer and can be coupled to the base portion 32. The transducer can be any selected types of transducer suitable for generating vibrations or vibrational energy, such as for example a piezo electric transducer.

The base portion 32 can also optionally include the acoustic therapy unit 22 for generating an audio output. The audio output can include selected types of music or sound. Further, the acoustic therapy unit can provide audio output that includes one or more of operating instructions for the system, providing mediation instructions for the person, providing breathing exercise instructions, and providing micro-movement therapy instructions. The music can help improve or affect behavior, motor function, social skills, emotions, coordination, self-expression and personal growth. The acoustic therapy unit 22 can also provide an audio output to help guide the subject through one or more meditative sessions or exercises. The acoustic therapy unit can also be located separate from the therapy bed but can be connected to and controlled thereby. The acoustic therapy unit can also include wireless capabilities to connect with headphones, audio devices or other separate acoustic components. In one embodiment, the acoustic therapy unit can be disposed within the base portion 32 or can be distributed between the top portion 36 and the base portion 32.

The base portion 32 can also include the top surface 42A that can employ a separate and optional radiation therapy unit 18 for applying radiation or light energy to the subject. The radiation therapy unit 18 can employ a series of LEDs for emitting light energy in specific wavelengths or wavelength bands. According to one embodiment, the radiation therapy unit 18 can include selected LEDs for generating radiation or light energy in the blue, red, and/or infrared wavelength ranges. The radiation therapy unit 16 can hence include LEDs configured for emitting energy in one of these wavelength ranges or in any combination of these wavelength ranges. For example, the LEDs can emit light energy in the infrared wavelength range, including for example in the near infrared, mid infrared, and far infrared wavelength ranges. The LEDs can also be individually adjusted to emit light at different frequencies. The emitters can emit radiation in wavelength ranges similar to or different from the radiation therapy unit 18 formed in the top portion 36. Thus, if dual radiation therapy units 18 are employed, then the radiation therapy units can be controlled to operate in conjunction with each other, such that they apply radiation energy to the subject at the same or different times and in the same or different wavelength ranges. The radiation energy can be continuous or pulsed or be some combination of the two, for instance LEDs emitting NIR light could be pulsed rather than continuous to aid in the recovery of the subject. The base portion can also be configured to include one or more cooling units 76, such as fans, for providing cooling and ventilation to the interior chamber and for cooling the chamber 44. The top surface 42A can also be optionally configured to include or support the bed portion 34.

The therapy bed can also include the bed portion 34. The bed portion 34 is an optional portion and can be included if the top surface 42A of the base portion is not employed as the bed portion. The bed portion 34 can thus form part of the top surface 42A of the base portion or can be supported by the base portion 32. The bed portion 34 can also be optionally spatially separated from the base portion by suitable support structure to form the intermediate bed portion. The illustrated bed portion 34 can include at least a portion of the electromagnetic therapy unit 18 for applying electromagnetic energy to the subject. As shown for example in FIGS. 3, 4 and 6A and 6B, the electromagnetic therapy unit 18 can employ one or more electromagnetic coils that are mounted on or in the bed portion 34. According to one embodiment, the bed portion 34 can include the coils 18A-18C, such as for example PEMF type coils (e.g., Hemholtz coils). The coils can be electrically coupled together in any selected arrangement, such as in series or in parallel. The electromagnetic therapy unit 18 can be, for example, a pulsed electromagnetic field therapy (PEMFT) unit, also known as a low field magnetic stimulation (LFMS) system or unit, that uses electromagnetic fields to heal portions of the body. The coils can be stationary or movable. The coils can generate electromagnetic energy in any selected wavelength or frequency range, such as in a low frequency range under 50 Hz. The coils can function as an antenna and/or as a circuit and can be pulsed or placed into a continuous operating mode. The coils can generate and direct pulsed electromagnetic energy waves toward the person and/or can be used as one or more antennas to generate one or more electromagnetic fields. The electromagnetic energy (e.g., electromagnetic field) generated by the coils can help bring the magnetic field of the subject back to their natural state. This can provide a wide range of benefits including improving ones well-being and synergistically working with infrared therapy. The illustrated intermediate bed portion 34 can be formed as a single molded component that can be planar or flat to form a flat platen surface or can be bent to form different sections. Alternatively, the bed portion 34 can be formed of multiple different components that are coupled together. According to the current embodiment, the bed portion 34 is a single molded component that has a main body 52 having a first end portion 54A, a second opposed end portion 54C, and an intermediate portion 54B that is disposed between and couples together the end portions 54A, 54C. The intermediate portion 54B can be a bent portion that places the portions on different planes. The intermediate bed portion 34 can be formed from any suitable material, such as for example from acrylic. The bed portion can be transparent or optically transparent and can be formed of any suitable material, such as acrylic. As used herein, the term “transparent” is intended to mean that a substantial portion of light or radiation energy is transmissible or passes through the bed portion. The term is intended to include forming the bed portion from any suitable material that is transparent, translucent, semi-transparent, opaque, and the like. Examples of suitable material include acrylic and other types of material that are sufficient to support the person.

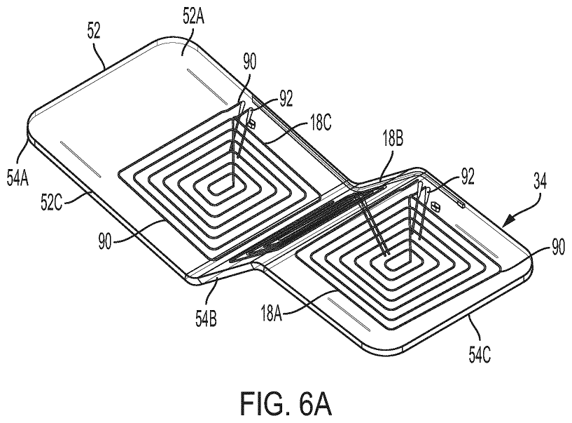

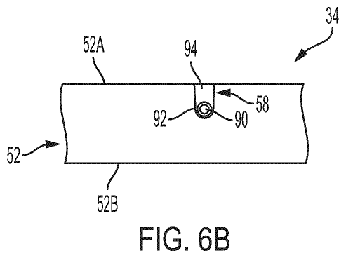

As shown in FIGS. 6A and 6B, the main body 52 of the bed portion 34 can have a top surface 52A and an opposed bottom surface 52B, as well as a side surface 52C that circumscribes the main body. The coils 18A-18C of the electromagnetic therapy unit 18 can be composed of one or more wires 90 that can be coupled to a power source. The wires can be stranded or solid and can have any cross-sectional shape, such as round or rectangular. The wires 90 can be of any length and gauge, and the coil can have any selected shape or configuration, such as a square or round shape and can be arranged in any selected pattern, such as in a concentric or non-concentric pattern. The power source can be mounted in the base portion 32 or the top portion 36. The main body 52 of the bed portion 34 can have one or more grooves 58 formed in the top or bottom surface to accommodate a portion of the wire 90 of the coils. The number of grooves can correspond to the number of coils formed in or on the bed portion 34. As shown by way of simple example, in FIG. 6B, the groove 58 is formed in the top surface 52A of the main body 52 so as to seat a portion of the wire 90. The groove 58 can have any selected type of shape, and can have a tapered configuration to form a friction fit when the wire 90 is inserted therein. The wire 90 can have an insulation or protective coating or layer 92 formed thereon. A fill layer 90 can be employed to fill the groove 58 such that when the material hardens, the wire 90 is retained within the groove. The fill layer 94 can employ any suitable material, such as an epoxy material. The edge surface 52C of the main body 52 can also have a selected groove formed therein to seat a selected light strip (not shown). The light strip when actuated can generate light in any selected color to create soothing, ambient lighting. Alternatively, the light strip can be attached or affixed to the edge surface 52C, or the light strip can be located anywhere on or within the bed main body, or anywhere that enables the light to shine therethrough, be reflected by, or be directed by the bed portion 34.

The bed portion 34 can be formed as a single unitary portion that can be relatively flat. Alternatively, the main body 52 of the bed portion 34 can be bent by any suitable techniques into the illustrated configuration, thus forming the bed portions 54A-54C. According to one practice, the main body 52 can be heated using any selected localized heating technique to soften one or more areas or regions of the main body, thus making the heated area pliable or malleable relative to the unheated portions. The main body can thus be subjected to heat of a sufficient temperature and for a sufficient period of time to soften the desired areas of the bed portion. The heated area can then be shaped or bent into the desired shape. Alternatively, the entire bed portion can be heated and then shaped.

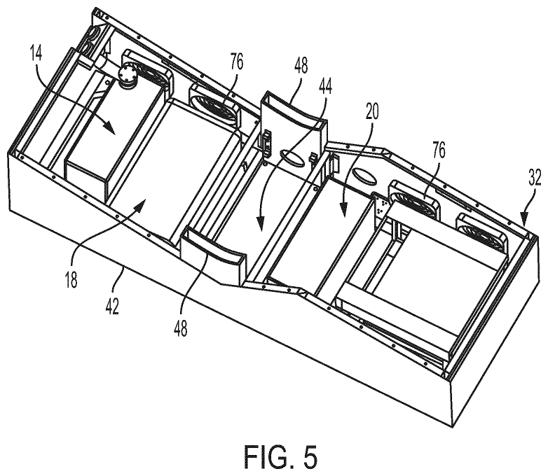

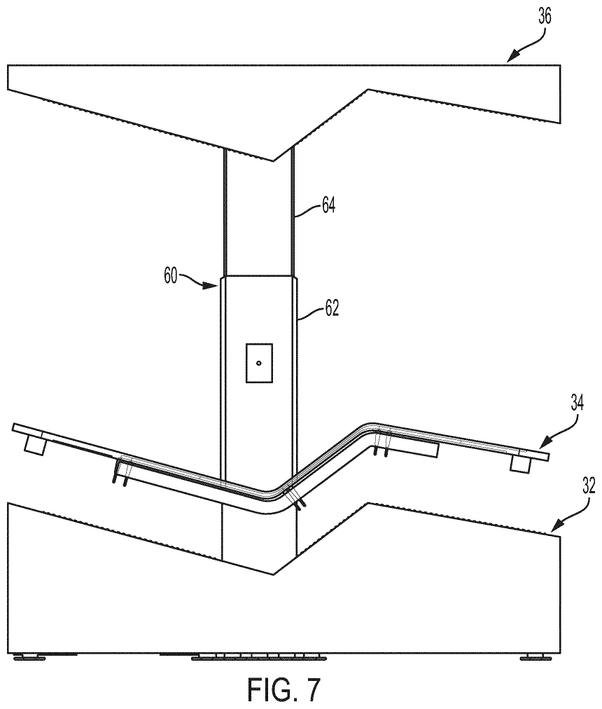

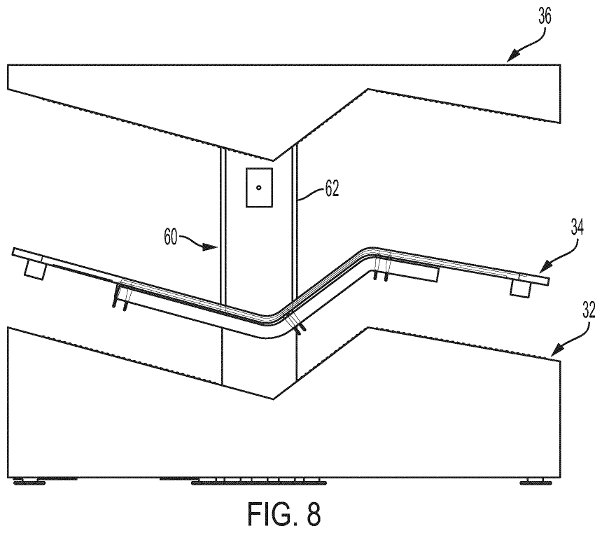

The bed portion 34 can be supported on the base portion 32 by any suitable support structure. According to one embodiment, the bed portion 34 is supported by a pair of opposed support elements 48, FIG. 5. The intermediate bed portion 34 can be stationary or can be movable relative to the top or bottom portions. Further, the top portion 36 of the therapy bed can be movable and operable relative to the base portion 32 and the intermediate bed portion 34. The top portion 36 can be supported by a support stanchion 60 that mounts and supports the top portion 36 relative to the bottom portion. The support stanchion 60 can allow the top portion to pivot so as to provide sufficient clearance or space to allow the subject to enter and to lie or sit on the bed, or is movable or translatable in the horizontal or vertical direction. According to one embodiment, the support stanchion 60 is configured to move the top portion 36 in the vertical direction. For example, as shown in FIGS. 7 and 8, the support stanchion 60 can include multiple support elements including an outer support element 62 and an inner telescoping support element 64. The support elements 62, 64 can be coupled to and driven by any suitable drive element, such as by a motor. The motor can be housed, for example, in the chamber 44 of the base portion 32. When the top portion 36 is moved vertically upwardly to an uppermost position, the top portion 36 is sufficiently spaced apart from the base portion 32 and the intermediate bed portion 34 to allow the subject to enter the therapy bed and to lie down on the bed portion 34. In this open position, as shown for example in FIG. 7, the inner support element 64 is extended outwardly from the outer support element 62 to a vertically maximal extent. After the subject is positioned on the bed portion 34, the top portion 32 can be lowered into a closed, operative position, as shown for example in FIG. 8. In the closed position, the inner support element 64 is disposed nearly completely or completely within the outer support element 62. The closed and/or open position can also be varied, adjusted or customized based on the subject's size, height, girth and/or shape. The support stanchion 60 can include an optional start button or switch 68 for allowing the user to activate the drive mechanism of the support stanchion 60 so as to move the top portion 36 between the open and closed positions. The start button or switch 68 can also function as a stop button, or these buttons can be separated and located anywhere on or off of the system.

The system 10 can also include one or more of a radionics therapy unit, a cranial stimulation therapy unit, an infrared sauna therapy unit, and a light therapy unit, such as a Lucia light therapy unit. The controller 24 controls the operation of the various features and units of the system 10. The user interface 26 allows a user to selectively operate one or more of the therapy units. The multi-function therapeutic system 10 can also be configured as a portable device that can be coupled or attached to a portion of a person for localized treatment of the person. Further, the base portion 32 of the system can be configured to have one or more slideable drawer units that allows the user to gain access to one or more specific therapy units that are mounted therein.

Those of ordinary skill in the art will readily recognize that selected components of the various therapy units of the present invention can be distributed throughout the system or located in portions different than the primary location of the active or therapeutic energy applying component. For example, the power sources of the radiation therapy units of the present invention can be located at different locations. In one embodiment, the power source of the radiation therapy unit located in the top portion can be located in the base or bottom portion.

According to other embodiments, the hydrogen therapy unit 14 can optionally include water or gas cleaning components, such as for example an ultraviolet light cleaning subsystem. The subsystem can include for example a pump to circulate the water in an associated storage tank past an ultraviolet light to destroy or eliminate bacteria and viruses. Alternatively, the subsystem can pass the hydrogen gas by an ultraviolet light before it is supplied to the person.

According to still other embodiments, the electromagnetic therapy unit 18 can include one or more field enhancement accessories that can be coupled thereto. The accessory can be configured to magnify the strength of the electromagnetic field when connected to the electromagnetic therapy unit 18 and held by, or otherwise touches, the person. For example, the accessory can be a hand-held electromagnetically-charged neon-filled glass ball.

It will thus be seen that the invention efficiently attains the objects set forth above, among those made apparent from the preceding description. Since certain changes may be made in the above constructions without departing from the scope of the invention, it is intended that all matter contained in the above description or shown in the accompanying drawings be interpreted as illustrative and not in a limiting sense.

It is also to be understood that the following claims are to cover all generic and specific features of the invention described herein, and all statements of the scope of the invention which, as a matter of language, might be said to fall therebetween.

We claim:

1. A multi-function therapeutic system, comprising a housing having
a top portion having a first radiation therapy unit mounted therein for generating and applying light energy to a person,
a bottom portion including a hydrogen therapy unit for supplying hydrogen to the person, and
an intermediate bed portion disposed between the top portion and bottom portion, wherein the intermediate bed portion includes an electromagnetic therapy unit for generating and applying electromagnetic energy to the person,
a vibration therapy unit associated with one or more of the bottom portion and the intermediate bed portion for generating and applying vibrational energy to the person, and
a control unit for controlling the first radiation therapy unit, the hydrogen therapy unit, the electromagnetic therapy unit, and the sound therapy unit.

2. The system of claim 1, wherein the housing is configured as a therapy bed.

3. The system of claim 2, wherein one or more of the bottom portion, the bed portion and the top portion further comprises an acoustic therapy unit for generating an audio output.

4. The system of claim 1, wherein the vibration therapy unit includes a first vibration generating sub-system coupled to the bottom portion for generating a first vibrational energy and a second vibrational subsystem coupled to the intermediate bed portion for generating a second vibrational energy.

5. The system of claim 4, wherein the first vibration generating sub-system includes a sub-woofer and the second vibration generating sub-system includes one or more transducers.

6. The system of claim 1, wherein the vibration therapy unit can be configured to operate as an acoustic therapy unit to generate an audio output.

7. The system of claim 4, wherein the bottom portion comprises a second radiation therapy unit for generating and applying light energy to the person.

8. The system of claim 7, wherein each of the first and second radiation therapy units generates light energy in a blue wavelength range, a red wavelength range, or an infrared wavelength range.

9. The system of claim 8, wherein each of the first and second radiation therapy units generates light energy in one or more of a near-infrared wavelength range, a mid-infrared wavelength range, and a far-infrared wavelength range.

10. The system of claim 4, wherein the electromagnetic therapy unit generates the electromagnetic energy in a continuous or a non-continuous manner.

11. The system of claim 10, wherein the electromagnetic therapy unit comprises one or more pulsed electromagnetic field (PEMF) coils for generating the electromagnetic energy.

12. The system of claim 4, wherein the controller controls movement of the top portion relative to the bottom portion between an open position and a closed position.

13. The system of claim 12, wherein the top portion is coupled to the bottom portion via a support stanchion.

14. The system of claim 13, wherein the support stanchion comprises an outer support element and an inner support element disposed within the outer support element, wherein the inner support element is configured for vertical movement within the outer support element.

15. A multi-function therapeutic system, comprising a housing having
a top portion having a first radiation therapy unit mounted therein for generating and applying radiation energy to a person,
a bottom portion having
a hydrogen therapy unit for supplying hydrogen to the person, and
a second radiation therapy unit for generating and applying radiation energy to the person,
a bed portion supported by the bottom portion, and
a control unit for controlling the first radiation therapy unit, the second radiation therapy unit, and the hydrogen therapy unit.

16. The system of claim 15, wherein the bed portion comprises an electromagnetic therapy unit for generating and applying electromagnetic energy to the person.

17. The system of claim 16, wherein the bottom portion further comprises a vibration therapy unit for generating and applying vibrational energy to the person.

18. A method for applying one or more therapeutic techniques to a person, comprising
providing a housing having a bottom portion, a top portion, and a bed portion disposed between the bottom portion and the top portion,
providing a first radiation therapy unit mounted in the top portion for generating and applying light energy to a person,
providing in the bottom portion a hydrogen therapy unit for supplying hydrogen to the person, and a vibration therapy unit for generating and applying vibrational energy to the person,
providing in the bed portion an electromagnetic therapy unit for generating and applying electromagnetic energy to the person, and
controlling the first radiation therapy unit, the hydrogen therapy unit, the electromagnetic therapy unit, and the sound therapy unit.

19. The method of claim 18, further comprising providing in the bottom portion a second radiation therapy unit for generating and applying light energy to the person.

20. The method of claim 19, further comprising generating with one or more of the first and second radiation therapy units light energy in one or more of a near-infrared wavelength range, a mid-infrared wavelength range, or a far-infrared wavelength range.

21. The method of claim 20, further comprising controlling movement of the top portion relative to the bottom portion between an open position and a closed position.

22. The method of claim 21, further comprising providing in one or more of the bottom portion and the top portion an acoustic therapy unit for generating an audio output.

23. A multi-function therapeutic system, comprising a housing having
a top portion having a first radiation therapy unit mounted therein for generating and applying light energy to a person,
a bottom portion including a second radiation therapy unit mounted therein for generating and applying light energy to the person, wherein the bottom portion supports a transparent bed portion having an electromagnetic therapy unit associated therewith for generating and applying electromagnetic energy to the person via one or more coils, and
a control unit for controlling the first radiation therapy unit, the second radiation therapy unit, and the electromagnetic therapy unit.

24. The system of claim 24, wherein the bed portion is spatially separated from the base portion to form an intermediate bed portion disposed between the base portion and the top portion.

25. The system of claim 23, wherein the housing further comprises one or more of
a hydrogen therapy unit for supplying hydrogen to the person, and
a vibration therapy unit for generating and applying vibrational energy to the person.

26. The system of claim 25, wherein the housing further comprises an acoustic therapy unit for generating an audio output.

27. The system of claim 25, wherein one or more of the first and second radiation therapy units generates light energy in one or more of a near-infrared wavelength range, a mid-infrared wavelength range, and a far-infrared wavelength range.

28. The system of claim 25, wherein the electromagnetic therapy unit generates the electromagnetic energy in a continuous or a non-continuous manner.

29. The system of claim 28, wherein the electromagnetic therapy unit comprises one or more pulsed electromagnetic field (PEMF) coils for generating the electromagnetic energy.

30. The system of claim 25, wherein the controller controls movement of the top portion relative to the bottom portion and the bed portion between an open position and a closed position.

31. A multi-function therapeutic system, comprising a housing having
an acoustic therapy unit for providing an audio output,
a vibration therapy unit for generating and applying vibrational energy to a person, and
at least two or more of:
a first radiation therapy unit for generating and applying light energy to the person,
a second radiation therapy unit for generating and applying light energy to the person,
an electromagnetic therapy unit for generating and applying electromagnetic energy to the person, and
a hydrogen therapy unit configured to supply hydrogen to the person.

32. The system of claim 31, wherein the audio output of the acoustic therapy unit comprises one or more of operating instructions for the system, providing mediation instructions for the person, providing breathing exercise instructions, and providing micro-movement therapy instructions.

33. A multi-function therapeutic system, comprising a housing having
a transparent bed portion forming a support surface for a person, wherein the bed portion has a main body having one or more coils coupled thereto for generating and applying electromagnetic energy to the person, wherein the one or more coils include one or more wires having an insulating layer formed over at least a portion of the one or more wires, and
a base portion having one or more support elements for supporting the transparent bed portion and having a first radiation therapy unit for generating and applying light energy to the person, wherein the light energy passes through the transparent bed portion to the person.

34. The system of claim 33, wherein the housing further comprises a top portion movable relative to one or more of the bed portion and the base portion.

35. The system of claim 34, wherein the top portion comprises a second radiation therapy unit mounted therein for generating and applying light energy to the person.

36. The system of claim 34, wherein the first radiation therapy unit generates light energy in one or more of a near-infrared wavelength range, a mid-infrared wavelength range, and a far-infrared wavelength range.

37. The system of claim 33, wherein the one or more coils comprises one or more pulsed electromagnetic field (PEMF) coils for generating the electromagnetic energy.

38. The system of claim 33, wherein the main body of the transparent bed portion further comprises a first bed portion, a second bed portion and a third bed portion, wherein the second bed portion is disposed between and is angled relative to the first and third bed portions forming multiple different planes.

39. A method of forming a transparent bed portion of a multi-function therapeutic system, comprising,
embedding at least one electromagnetic field generation coils within a support surface of the transparent bed,
forming at least one groove in the support surface that are sized and configured for seating one or more wires of the one or more electromagnetic field generation coils,
inserting the wire in the groove to form a friction fit,
applying a selected hardening material to the groove to secure the wire in the groove, heating one or more portions of the support surface to a selected temperature such that the heated portion is pliable, and
bending the one or more heated portions of the support surface to form one or more angled sections.

40. The method of claim 39, further comprising coupling one or more transducers to the support surface to generate vibrational energy.

41. The method of claim 40, further comprising coupling one or more lights to the support surface.

42. The method of claim 41, further comprising applying a protective or insulative sheathing to the wire prior to insertion into the groove.
